(19) United States
(12) Patent Application Publication
(10) Pub. No.: US 2009/0209804 A1
(43) Pub. Date: Aug. 20, 2009

(54) Apparatuses and methods for percutaneously implanting objects in patients

(75) Inventors: Keith Seiler, Issaquah, WA (US); Eric Hadford, Snohomish, WA (US); Margo Gisselberg, Lynnwood, WA (US)

Correspondence Address: CALYPSO MEDICAL / PERKINS COIE, LLP, P.O. BOX 1247, SEATTLE, WA, 98111-1247, US

(73) Assignee: Calypso Medical technologies, Inc., Seattle, WA (US)

(21) Appl. No.: 11/658,331

(22) PCT Filed: Jul. 25, 2005
(86) PCT No.: PCT/US05/26438

§ 371 Date: Dec. 3, 2008

Related U.S. Application Data
(60) Provisional application No. 60/590,521, filed on Jul. 23, 2004.

(51) Int. Cl. A61M 36/12; G08B 1/08

(52) U.S. Cl. 600/7; 340/539.12

(57) ABSTRACT

Apparatuses and methods for percutaneously implanting objects, such as radioactive seeds or markers, in patients. In one embodiment, a device for percutaneously implanting an object in a patient includes a handle, a cannula projecting outwardly from the handle, and an actuator movably disposed relative to the handle. In one aspect of this embodiment, the cannula can be configured to releasably hold the object and percutaneously penetrate the patient. In another aspect of this embodiment, the actuator can be operably connected to the cannula and operable to move the cannula relative to the handle and release the object within the patient. In a further aspect of this embodiment, the cannula can include a tip portion having a restriction configured to releasably hold the object for implantation in the patient.

### RELATED APPLICATIONS

This application is a continuation-in-part of application Ser. No. 10/334,699, filed on Dec. 30, 2002. This application claims the benefit of and priority to U.S. provisional patent application no. 60/590,521 filed on Jul. 23, 2004, which is hereby incorporated in its entirety herein by reference.

### TECHNICAL FIELD

The following disclosure relates generally to medical devices for percutaneously implanting markers or other small objects in patients.

### BACKGROUND

A number of existing medical treatments involve percutaneously inserting or implanting objects in a patient. One such treatment is brachytherapy for prostate cancer. In brachytherapy, radioactive sources or “seeds” are implanted relative to a tumor to provide a high dose of radiation to the tumor but not the surrounding healthy tissue. Other oncological treatments involve percutaneously implanting radio-opaque markers or signal-generating markers adjacent to the tumor. The markers identify the location of the tumor so that a high dose of radiation from a linear accelerator or other external source can be focused directly at the tumor.

FIGS. 1A and 1B are cross-sectional views of a two-piece introducer 100 of the prior art. Referring first to FIG. 1A, the introducer 100 includes a needle 102 and a stylet 104 slidably disposed within the needle 102. The stylet 104 includes a first handle 101 and a blunt distal end 106. The needle 102 includes a second handle 103 and a cannula 108 extending through the second handle 103. The cannula 108 is configured to hold radioactive seeds 110 or other objects. The cannula 108 has a distal tip 105 configured to percutaneously penetrate the patient for Implantation of the seeds 110 in the patient. Inert spacers 111 can be used to provide the desired spacing between the seeds 110 when the seeds 110 are implanted in the patient. The seeds 110 and spacers 111 are retained in the cannula 108 by a plug 112 made from bone wax or other suitable bio-compatible materials.

To implant the seeds 110 at a target location in a patient (not shown) in the desired pattern as loaded in the cannula 108, an operator (also not shown) pushes the cannula 108 in a first direction 120 to insert the tip 105 into the patient. The operator then pushes the second handle 103 further in the first direction 120 to position the tip 105 at the desired depth within the patient where the seeds 110 are to be released. Throughout this motion, the operator moves the needle 102 and the stylet 104 together as a unit. At the desired depth, the operator grasps the first handle 101 with one hand and the second handle 103 with the other hand and, while holding the first handle 101 stationary, slides the second handle 103 back in a second direction 122 toward the first handle 101. As shown in FIG. 1B, this movement causes the cannula 108 to pull back from the plug 112, the seeds 110, and the spacers 111 to implant them in the patient.

One shortcoming of the prior art introducer 100 is that the two-handed movement required to properly release the seeds 110 at the target location and in the desired pattern may be somewhat awkward and nonintuitive. As a result, the operator is prone to err and may inadvertently misplace the seeds 110. For example, to properly release the seeds 110, the operator must hold the first handle 101 stationary while sliding the second handle 103 back in the second direction 122 toward the first handle 101. If, instead, the operator accidentally pushes the first handle 101 toward the second handle 103, then the stylet 104 may push the seeds 110 out of the cannula 108 in the first direction 120. This movement could cause the seeds 110 and the spacers 111 to collide in a “train wreck” just beyond the tip 105 of the cannula 108. Either way, the seeds will not be positioned accurately relative to the target location or in the desired pattern. A further shortcoming of the prior art introducer 100 is that the bone wax used for the plug 112 in brachytherapy applications may melt prematurely allowing the seeds 110 to migrate out of the cannula 108 before reaching the desired target location. As such, conventional introducers for brachytherapy applications are custom loaded at the treatment facility and are not suitable for being transported in warm environments.

Markers that transmit a signal pose additional challenges for introducers. In the case of markers with magnetic transponders or other radio frequency transmitters, it is desirable to check the functionality and other attributes of the markers after loading the markers in the introducers but before implantation. Assuring functionality of a marker after packaging but before implantation reduces complications caused by implanting a nonfunctioning or a malfunctioning marker. Conventional introducers, which are made of metals and metal alloys, are not well suited for testing markers after the markers have been loaded in the cannulas because the electromagnetic waves emitted by the marker are absorbed by the metal cannulas. As such, even though the cannulas do not completely enclose the markers, they nevertheless can reduce the signal strength outside of the cannulas. Thus, conventional introducers may prevent testing a marker while it is in the introducer.

### SUMMARY

The invention is directed to apparatuses and methods for implanting markers, radioactive seeds, radio frequency transponders, or other small objects in patients. In one aspect, a device for percutaneously implanting an object in a patient includes a handle, a cannula projecting outwardly relative to the handle, and an actuator operably connected to the cannula and movably disposed relative to the handle. The cannula can have a proximal portion positioned proximate to the handle and a distal portion configured to releasably hold the object and percutaneously penetrate the patient by movement of the handle. The actuator can be operable to slide the cannula relative to the handle and release the object within the patient.

In another aspect, the device can further include a stylet extending at least partially within the cannula and being fixedly positioned with respect to the handle. Operating the actuator to slide the cannula relative to the handle causes the cannula to slide relative to the stationary stylet and release the object within the patient.

In a further aspect, the cannula can include a tip portion having a restriction configured to releasably hold the object for implantation in the patient, and the actuator can be selectively movable from a first position to a second position. When the actuator is in the first position, the tip portion of the cannula can at least generally retain the object. When the actuator is in the second position, the cannula can be drawn back from the object to overcome the restriction and release the object within the patient.

In yet another aspect, a method for percutaneously implanting an object in a patient includes moving a handle to percutaneously insert a cannula projecting from the handle within the patient, and moving the cannula relative to the handle to release the object within the patient. Moving the cannula relative to the handle can include sliding the cannula with respect to a stationary stylet extending coaxially through at least a portion of the cannula. Moving the handle to percutaneously insert the cannula can include driving the handle forward with a hand of an operator. Further, moving the cannula relative to the handle to release the object within the patient can include manipulating an actuator with a digit of the hand of the operator to move the cannula aft relative to the handle while the handle remains stationary in the hand of the operator.

Still another aspect of the invention is directed to an introducer assembly that enables markers with alternating magnetic transponders to be tested while they are assembled with or otherwise loaded in the introducer. In one embodiment, the introducer includes a cannula, a stylet configured to fit in the cannula, and a holder configured to releasably retain a marker. The holder is further configured to allow a sufficient signal level from the marker to propagate from the introducer for testing the marker after it has been assembled with the introducer but before implanting the marker. The holder can be a dielectric housing at a proximal end and/or distal end of the cannula. For example, the holder can include a retaining element that engages a peripheral surface of the marker to retain the marker within the holder. In an alternative embodiment, the holder is a distal portion of the cannula having a hole through the cannula wall. The hole through the cannula wall is large enough to allow a sufficient portion of the electromagnetic signal transmitted from the marker to pass through the cannula wall and be measured by a sensor.

In operation, the marker is initially loaded into the holder for testing. The marker is tested by generating an alternating magnetic excitation field at the resonant frequency of the marker, terminating the excitation field, and sensing a wirelessly transmitted alternating magnetic location signal from the marker. Because the holder is a dielectric housing or a portion of the cannula having holes through the cannula wall, a sufficient amount of energy from the location signal propagates to a sensor. As a result, the markers can be tested after they have been loaded into the introducers to insure that the markers operate before shipping the loaded introducers. Additionally, the markers can also be tested just before they are implanted into the patient to further insure that only operable markers are implanted.

### BRIEF DESCRIPTION OF THE DRAWINGS

FIGS. 1A and 1B are cross-sectional views of a two-piece introducer of the prior art.

FIGS. 2A and 2B are hidden isometric views of an introducer in accordance with an embodiment of the invention with a distal portion of the introducer shown in cross-section.

FIG. 3 is a cut-away isometric view of the Introducer shown in FIGS. 2A and 2B in accordance with an embodiment of the invention with a portion of the introducer shown in cross-section.

FIGS. 4A and 4B are enlarged hidden side and bottom views, respectively, of a tip portion of a cannula in accordance with an embodiment of the invention.

FIGS. 5A-C are enlarged cross-sectional views of the introducer shown in FIGS. 2A and 2B illustrating operation of an actuator in accordance with embodiments of the invention.

FIG. 6 is a hidden isometric view of an introducer in accordance with another embodiment of the invention with a distal portion of the introducer shown in cross-section.

FIGS. 7 is a cross-sectional isometric view of an introducer having an external actuator in accordance with another embodiment of the invention.

FIG. 8 is an isometric view of an introducer with a button having a flange extending over the handle to restrict the button from being pushed too far into the handle in accordance with another embodiment of the invention.

FIGS. 9A, 9B and 9C are cross-sectional views showing a portion of an introducer having a pivoting actuator in accordance with yet another embodiment of the invention.

FIGS. 10A and 10B are cross-sectional views of an introducer having a trigger-operated actuator in accordance with yet another embodiment of the invention.

FIG. 11 is a cross-sectional view of an introducer in accordance with yet another embodiment of the invention.

FIGS. 12A, 12B and 12C are cross-sectional views of introducers including retainers that restrict the stylets from moving with respect to the cannulas in accordance with additional embodiments of the invention.

FIG. 12D is an isometric view of an introducer including a retainer in accordance with another embodiment of the invention.

FIG. 13 is an isometric view of an introducer having a holder with holes through the cannula wall for testing a marker in accordance with an embodiment of the invention.

FIG. 14A is an isometric view of an introducer with a dielectric holder for testing a marker in accordance with yet another embodiment of the invention.

FIGS. 14B and 14C are cross-sectional views showing a portion of the introducer of FIG. 14A.

FIG. 15A is an isometric view of an introducer with another dielectric holder in accordance with yet another embodiment of the invention.

FIG. 15B is a cross-sectional view showing a portion of the introducer of FIG. 15A in greater detail.

FIG. 16 is an isometric view of an introducer and a marker container in a package assembly in accordance with another embodiment of the invention.

FIG. 17 is an enlarged, partially exploded isometric view of the introducer of FIG. 16 shown removed from the package assembly.

FIG. 18 is an enlarged, partially exploded isometric view of the marker container of FIG. 16 shown removed from the package assembly.

FIG. 19 is an enlarged cross-sectional view of the marker container (with a cap removed) connected to a proximal end of the introducer of FIG. 17.

### DETAILED DESCRIPTION

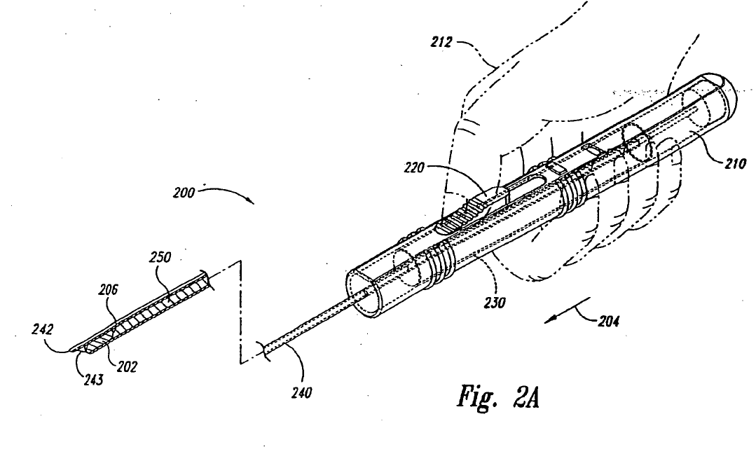

The following disclosure describes medical devices and methods for percutaneously implanting objects, such as radioactive seeds or markers, in patients. Certain specific details are set forth in the following description and in FIGS. 2A-15B to provide a thorough understanding of various embodiments of the invention. Certain well-known details often associated with such medical devices are not set forth in the following disclosure to avoid unnecessarily obscuring the various embodiments of the invention. Further, those of ordinary skill in the relevant art will understand that they can practice other embodiments of the invention without several of the details described below. In the drawings, identical reference numbers identify identical or at least generally similar elements.

FIGS. 2A and 2B are hidden isometric views of an introducer 200 in accordance with an embodiment of the invention with a distal portion of the introducer being cut-away. Referring first to FIG. 2A, one embodiment of the introducer 200 includes a handle 210, a hollow needle or cannula 240 projecting outwardly from the handle 210, and an actuator 230 fixedly attached to the cannula 240 and movably disposed within the handle 210. The cannula 240 has a proximal portion slidably disposed within the handle 210 and a distal tip portion 242. The cannula 240 can be a 14 gauge needle or smaller in many applications. The introducer 200 also includes a stylet 250 extending coaxially within the cannula 240. The stylet 250 can be fixedly attached relative to the handle 210 and can include a blunt distal end 206. The actuator 230 can include a button 220 manually operable to move the actuator 230 and the cannula 240 fore and aft with respect to the stylet 250 using a digit of a single hand.

A signal-generating marker 202, a radio-active seed or other implantable object is slidably positioned in the cannula 240 between the distal end 206 of the stylet 250 and the tip portion 242 of the cannula 240. The tip portion 242 can be configured to percutaneously penetrate the patient for implantation of the marker 202, and can include a restriction 243 configured to releasably retain the marker 202 in the cannula 240 prior to release of the marker 202 in the patient. In other embodiments, the cannula 240 can hold other objects for implantation in the patient in addition to the marker 202. For example, in another embodiment, the cannula 240 can hold additional markers optionally spaced apart by one or more spacers to provide a desired marker pattern. Similarly, in a further embodiment, the cannula 240 can hold a plurality of radioactive seeds optionally spaced apart by one or more spacers to provide a desired seed pattern.

To percutaneously implant the marker 202 in a patient (not shown), an operator 212 grasps the handle 210 in one hand and aligns the cannula 240 with a desired point of entry on the patient. The operator 212 then moves the handle 210 in a forward direction 204 to position the tip portion 242 of the cannula 240 at the target location within the patient (for example, proximate to a tumor). During this movement, the cannula 240 is held stationary relative to the stylet 250. Referring next to FIG. 2B, after the tip portion 242 is at the target location, the operator 212 uses a single hand to move the button 220 in an aft direction 207 relative to the handle 210 and hold the stylet 250 stationary relative to the handle 210. This movement draws the cannula 240 back in the aft direction 207 over the marker 202 and the stylet 250. The stylet 250 is fixed to the handle 210 and remains stationary so that the marker 202 is implanted in the patient as the cannula 240 moves aftward. The operator 212 can now move the handle 210 in the aft direction 207 to retract the cannula 240 from the patient.

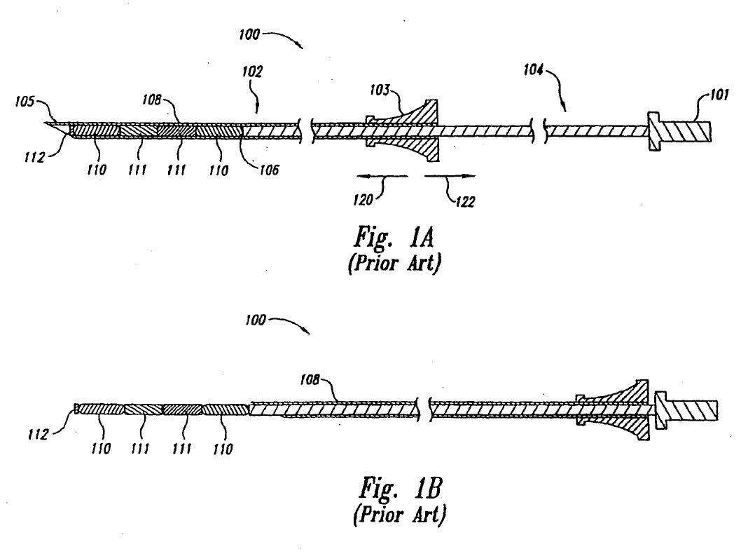

One feature of embodiments of the introducer 200 shown in FIGS. 2A and 2B is that the operator can accurately release the marker 202 in the patient by a single movement of a digit of one hand. More specifically, because the stylet 250 is fixed to the handle 210 and the actuator 230 is operated by the operator's hand that holds the handle 210, the stylet 250 cannot push the markers 202 out of the cannula 240. An advantage of this feature is that the required movement is intuitive and simple to execute, thus avoiding the possibility of driving the markers out of the cannula causing a “train wreck.” In contrast, the prior art introducer 100 of FIGS. 1A and 1B requires a potentially awkward two-handed movement to properly release objects at a target location within a patient. The intuitive movement of the prior art device is to move the handles 101 and 103 (FIG. 1A) toward each other. As a result, an operator of the prior art introducer 100 is prone to err and may inadvertently misplace the objects.

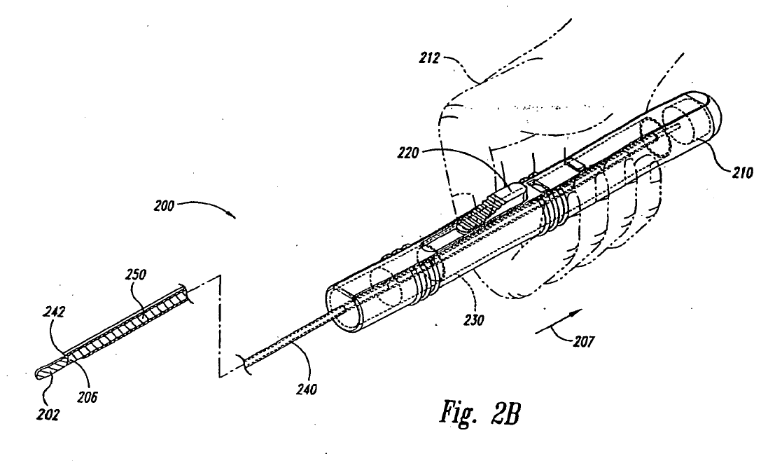

FIG. 3 is a cross-sectional isometric view of the introducer 200 shown in FIGS. 2A and 2B in accordance with an embodiment of the invention. In one aspect of this embodiment, the stylet 250 is fixedly attached to an end cap 360. The end cap 360 can include an engagement portion 362 configured to be received in a handle opening 312 in the handle 210. In the illustrated embodiment, the distal end 206 of the stylet 250 can be at least generally blunt. In other embodiments, the distal end 206 can have other shapes depending on the particular application. For example, in other embodiments, the distal end 206 can have a beveled, pencil-point shape, or full round shape.

In another aspect of this embodiment, the actuator 230 is at least generally hollow and includes a body 330, a bore 332 through the body 330, a position selector 334, and an opening 335 at one end of the body 330 opposite the bore 332. A proximal end of the cannula 240 is positioned in the bore 332 and fixedly attached to the body 330. The cannula 240 can extend from the opening 335 and project outwardly from the bore 332. The position selector 334 of the illustrated embodiment includes an indexing feature or protruding tab 338 and a button pad 337 for mounting the button 220. First and second slits 331 a and 331 b are positioned on opposite sides of the position selector 334 and allow the protruding tab 338 to deflect resiliently inward in response to depression of the button 220.

In a further aspect of this embodiment, the handle 210 is at least generally hollow and includes an interior portion 314 and a cannula opening 319. The interior portion 314 can be configured to slidably receive the actuator 230, and the cannula opening 319 can be configured to allow the cannula 240 to slide freely back and forth with respect to the handle 210 as the actuator 230 moves back and forth within interior portion 314 of the handle 210.

In yet another aspect of this embodiment, the handle 210 further includes a button opening 316 and locking features 318. In the illustrated embodiment, the locking features 318 include a first tab opening 318a and a second tab opening 318b. The locking features 318 can be configured to selectively receive the protruding tab 338 of the position selector 334 as the operator (not shown) moves the position selector 334 fore and aft in the handle 210 with the button 220. As will be explained in greater detail below, in other embodiments, the handle 210 can include more locking features depending on the number of markers 202 or other objects the introducer 200 is configured to implant.

The introducer 200 can be assembled by inserting the cannula 240 through the handle opening 312 and the cannula opening 319 until the button pad 337 is aligned with the button opening 316 and the protruding tab 338 engages the first locking feature 318a. The button 220 is then fixedly attached to the button pad 337. The marker 202 can then be inserted into the cannula 240 through a cannula inlet 343 at the proximal end of the cannula 240. In other embodiments, the marker 202 can be inserted into the distal end of the cannula. The cannula inlet 343 can be flared or otherwise configured for smooth loading of the marker 202 or other objects, such as seeds and/or spacers. The distal end 206 of the stylet 250 is then inserted into the cannula inlet 343 and moved through the cannula 240 driving the marker 202 through the cannula 240 until the engagement portion 362 of the cap 360 mates with the handle opening 312. At this point the marker 202 is releasably held in the cannula 240 between the distal end 206 of the stylet 250 and the restriction 243 of the tip portion 242.

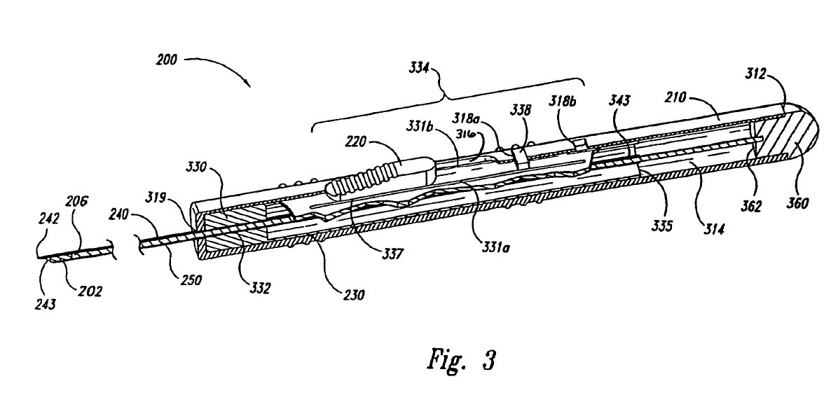

FIGS. 4A and 4B are enlarged hidden side and bottom views, respectively, of the tip portion 242 of the cannula 240 in accordance with an embodiment of the invention. Referring first to FIG. 4A, in one aspect of this embodiment, the tip portion 242 includes a beveled edge 460 configured to facilitate percutaneous penetration of the patient. In other embodiments, the tip portion 242 can have other configurations for facilitating percutaneous penetration. For example, in another embodiment, the tip portion 242 can include a double-beveled edge.

Referring now to FIG. 4B, in another aspect of this embodiment, the restriction 243 includes a first crimp 444a and a second crimp 444b formed in the beveled edge 460. The crimps 444 can be shaped and sized to reduce the width of the cannula 240 to be less than the diameter of the marker 202. This reduction in width can be tailored to provide a small resistance sufficient to retain the marker 202 in the cannula 240 until the tip portion 242 moves aft over the distal end 206 of the stylet 250. The restriction can be further tailored to provide the required resistance without scratching or otherwise damaging the marker 202 or, as the case may be, other objects such as radioactive seeds that can be implanted with the introducer 200 (FIGS. 2A and 2B). In other embodiments, other types of restrictions can be used to releasably retain the marker 202 in the cannula 240. For example, in another embodiment, the restriction can include only a single crimp on one side of the cannula 240. In a further embodiment, the restriction can include material added to the tip portion 242 proximate to the beveled edge 460, such as weld material or cured adhesive. In yet another embodiment, the restriction can include a feature machined or otherwise formed into the tip portion 242 proximate to the beveled edge 460.

One feature of embodiments of the invention shown in FIGS. 4A-B is that the restriction 243 is only slightly smaller than the outside diameter of the marker 202. An advantage of this feature is that the restriction 243 provides tactile feedback to the operator (not shown) as the tip portion 242 retracts over the marker 202. Such tactile feedback provides an indication to the operator that the marker 202 has been released within the patient. This feature can be advantageous when the introducer 200 is used to sequentially implant a plurality of objects, such as a plurality of markers, at different depths within the patient. Another feature of embodiments of the invention shown in FIGS. 4A-B is that the restriction 243 is positioned at least proximate to and often at the beveled edge 460. An advantage of this feature is that the beveled edge 460 provides a spring-back effect that further enhances the tactile feedback provided to the operator of the introducer 200.

Yet another feature of embodiments of the invention shown in FIGS. 4A-B is that the restriction 243 avoids the use of bone wax or other materials used in the prior art to hold the marker 202 in the cannula 240 prior to release. An advantage of this feature is that these other materials can melt or otherwise fail prematurely allowing the marker 202 to migrate out of the cannula 240 prior to reaching the target location. In contrast, the restriction 243 provides an environmentally stable solution that is not susceptible to fluctuating temperatures. Another advantage is that bone wax is not inadvertently introduced into a patient.

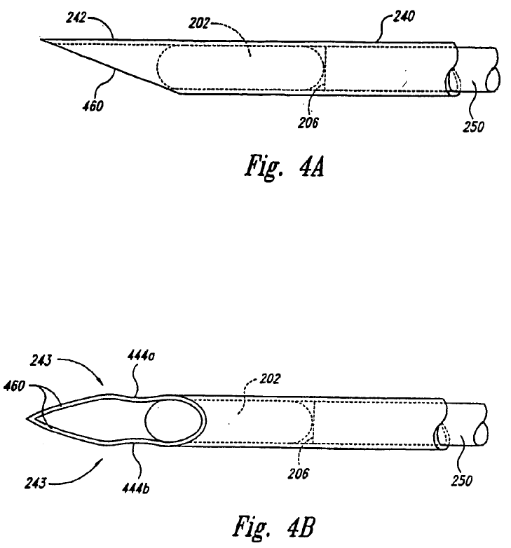

FIGS. 5A-C are enlarged cross-sectional views of the introducer 200 illustrating operation of the position selector 334 in accordance with embodiments of the invention. FIG. 5A shows the introducer 200 configured for insertion of the cannula 240 into the patient to implant the marker 202. In this mode, the protruding tab 338 of the position selector 334 engages the first locking feature 318a on the handle 210, thus holding the cannula 240 stationary relative to the stylet 250. In FIG. 5B, the tip portion 242 is at the target location within the patient and the operator 212 depresses the button 220 causing the protruding tab 338 to disengage from the first locking feature 318a. The operator 212 now moves the button 220 in the aft direction 207 sliding the actuator 230 aft in the handle 210. As shown in FIG. 5C, sliding the actuator 230 aft in the handle 210 draws the cannula 240 back over the stationary stylet 250 releasing the marker 202 in the patient. The operator 212 now releases the button 220 allowing the protruding tab 338 to engage the second locking feature 318b. The operator 212 can now retract the cannula 240 from the patient.

Those of ordinary skill in the relevant art will recognize that the structures described above for controlling the position of the cannula 240 relative to the stylet 250 (such as the position selector 334, the button 220, the protruding tab 338, and the locking features 318) represent but one embodiment of the present invention. Accordingly, in other embodiments, the features described above can have other details without departing from the spirit or scope of the invention. For example, in another embodiment, the protruding tab 338 and the locking features 318 can be omitted and the position of the actuator 230 can be manually controlled by the operator 212 or can be controlled by a friction surface, such as a serrated surface, existing between the actuator 230 and the handle 210.

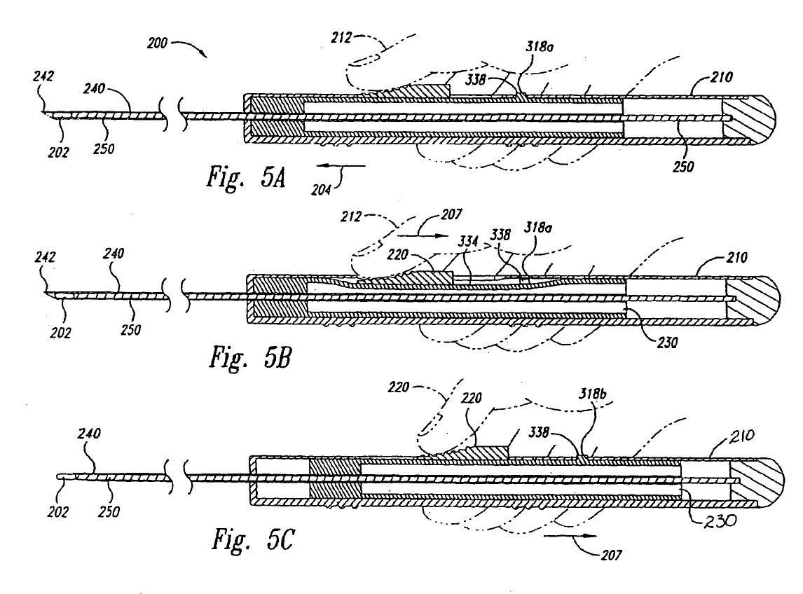

FIG. 6 is a hidden isometric view of an introducer 600 in accordance with another embodiment of the invention with a distal portion of the introducer shown cut-away. In one aspect of this embodiment, the introducer 600 includes a handle 610, a cannula 640 projecting outwardly from the handle 610, and an actuator 630 fixedly attached to the cannula 640 and movably disposed within the handle 610. The handle 610, the cannula 640, and the actuator 630 can be at least approximately similar in structure and function to their counterparts of the introducer 200 described above with reference to FIGS. 2A-5C. In another aspect of this embodiment, however, the handle 610 includes an elongated button opening 616 and a plurality of locking features 618 (shown as a first tab opening 618a, a second tab opening 618b, a third tab opening 618c, and a fourth tab opening 618d). The locking features 618 are configured to selectively receive a protruding tab 638 projecting from the actuator 630.

In another aspect of this embodiment, a plurality of markers 602 are slidably positioned in the cannula 640. Accordingly, an operator (not shown) can sequentially release the markers 602 in a patient (also not shown) by sequentially depressing a button 620 and moving the button 620 aft relative to the handle 610. With each aft movement, the protruding tab 638 is selectively received by one of the locking features 618. In this manner, the operator can monitor and control the timing of each marker release. The operator, for example, can implant a first marker 602 at a first target location, reposition the introducer 600, and implant a second marker 602 at a second location without having to reload the introducer 600.

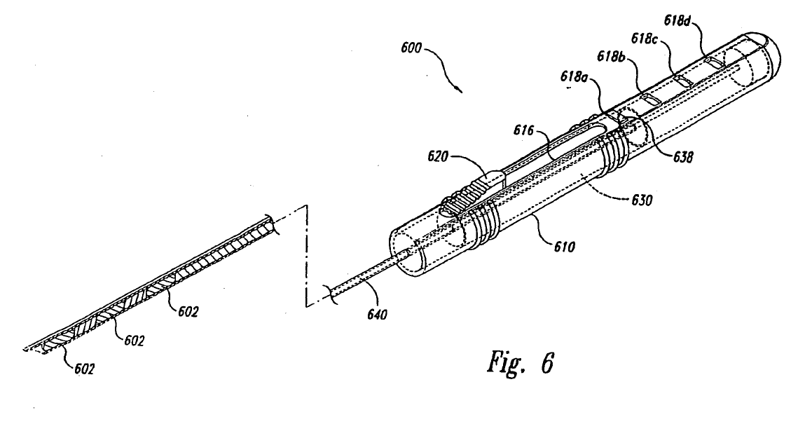

FIGS. 7 is a cross-sectional isometric view of an introducer 700 having an external actuator 730 in accordance with another embodiment of the invention. In one aspect of this embodiment, the introducer 700 includes a cannula 740 projecting outwardly relative to a handle 710. The cannula 740 is fixedly attached to the actuator 730, and the actuator 730 is slidably disposed over at least a portion of the handle 710. The introducer 700 further includes a stylet 750 fixedly attached to the handle 710 and extending coaxially within the cannula 740. In another aspect of this embodiment, the actuator 730 can include a rocker-button 720 with a protruding tab 738 configured to be selectively received in locking features 718 of the handle 710 (shown as a first locking feature 718a and a second locking feature 718b). Depressing the rocker-button 720 can disengage the protruding tab 738 from the first locking feature 718a and allow the actuator 730 to be slid aft in direction 707 relative to the handle 710. This action causes the cannula 740 to slide aft over the marker 202 and the stationary stylet 750 releasing the marker 202.

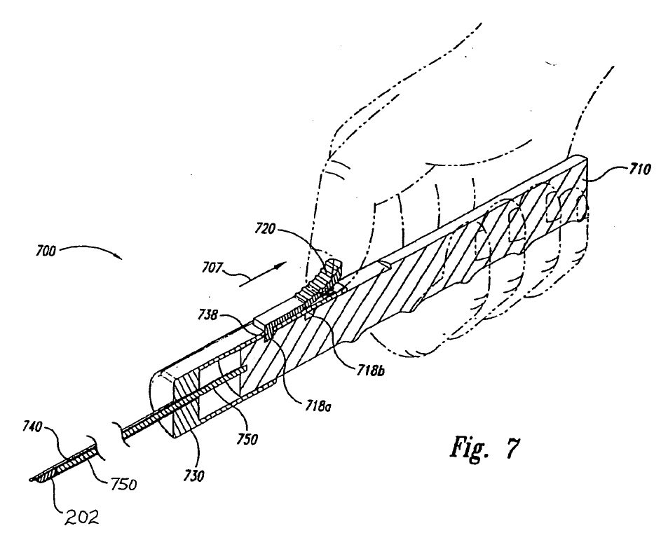

FIG. 8 is an isometric view of an introducer 800 in accordance with another embodiment of the invention. The introducer 800 is similar to the introducers shown in FIGS. 2 and 5, and thus like reference numbers refer to like components in these figures. In the introducer 800, the button 220 has an extension 810 over the handle 210 to prevent the button 220 from moving into the handle by more than a gap distance between the extension 810 and the handle 210. The extension 810 of the button 220 may be a flange protruding laterally from the sides of the button 220 to extend laterally beyond the sides of the slot in the handle 210. The extension 810 is spaced apart from the handle 210 when the button 220 is not depressed by a predefined distance. As such, regardless of the downward force exerted against the button 220, the extension 810 prevents the button 220 from traveling too far into the handle 210.

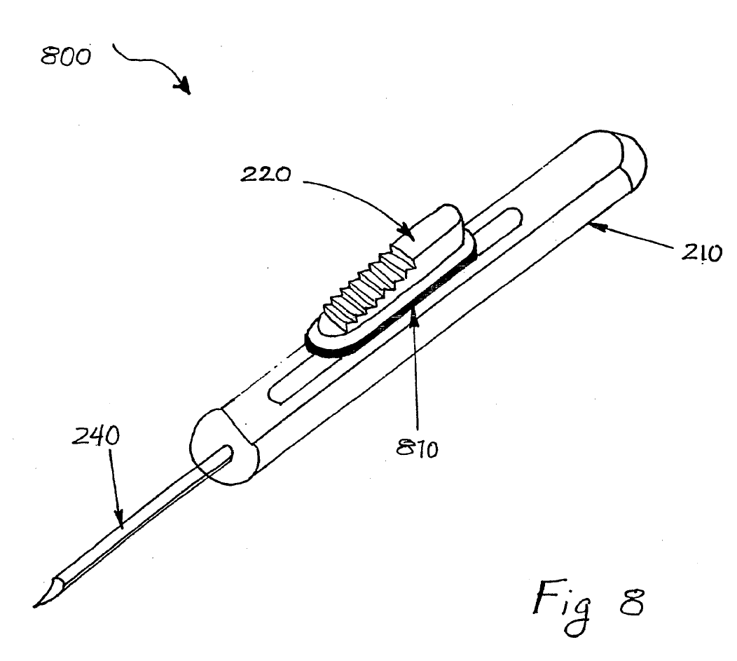

FIG. 9A is a cross-sectional view of an introducer 900 in accordance with yet another embodiment of the invention. The introducer 900 is also similar to the introducer 200 shown in FIG. 2, and thus like reference numbers refer to like components in these figures. The introducer 900 has a pivoting button 220 attached to the handle 210 at a pin 905. In this embodiment, the button 220 and the actuator 230 act as cam-and-follower, respectively. For example, the actuator 230 has a slot 910 including a first slanted face 911 and a second slanted face 912, and the button has a pin 920 received in the slot 910. The pin 920 is a cam, and the faces 911 and 912 of the slot are followers that translate the upward/downward movement of the button 220 to forward/backward movement of the actuator 230. In operation, as the button 220 moves downward, the pin 920 slides downward along the first face 911 of the slot 910 to drive the actuator 230 backwards further into the handle 210. Similarly, as the button 220 moves upward, the pin 920 slides upward along the second face 912 of the slot 910 to drive the actuator 230 forward. The introducer 900 can also include a spring 930 to urge the actuator 230 forward and consequently move the button 220 upward.

FIG. 9B illustrates an alternative embodiment of the introducer 900 in which the actuator 230 has a slanted face 940 and the button 220 has a contact element 950 contacting the face 940. FIG. 9C illustrates another alternative embodiment of the introducer 900 in which the button 220 has a contact element 960 with a slanted face 962 and the actuator 230 has a pin 970 contacting the face 962. In the embodiments of FIGS. 9B and 9C, a spring (not shown) can push the actuator 230 forward to move the button 220 upward.

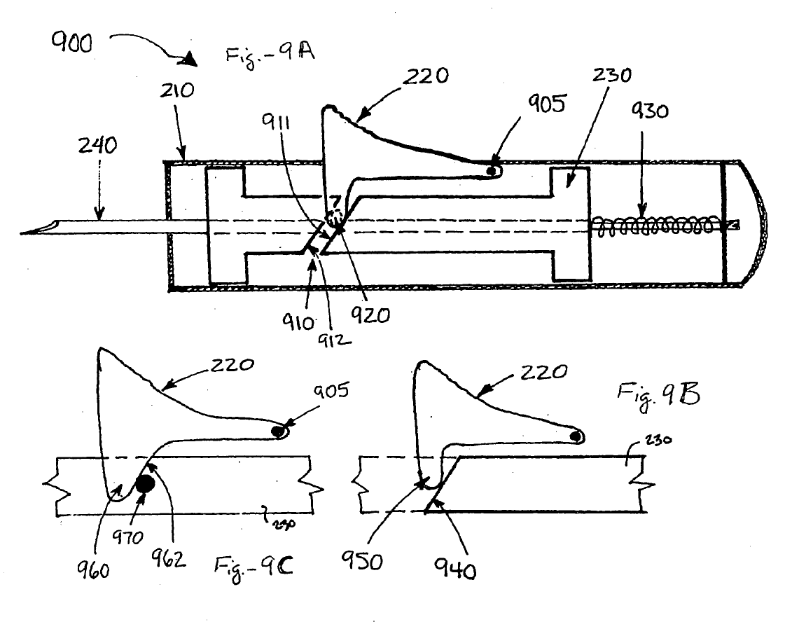

FIG. 10A is a cross-sectional view of an introducer 1000a in accordance with yet another embodiment of the invention. The introducer 1000a is similar to the introducer 900 shown in FIG. 9, and thus like reference numbers refer to like components in these figures. In the introducer 1000a, the button 220 is a trigger and the handle 210 is in the form of a pistol-grip. Pulling the trigger 220 in the embodiment of FIG. 10A performs the same function as depressing the button 220 in the embodiment of FIG. 9. FIG. 10B is a cross-sectional view of an introducer 1000b in accordance with yet another embodiment of the invention in which the button 220 is a lever.

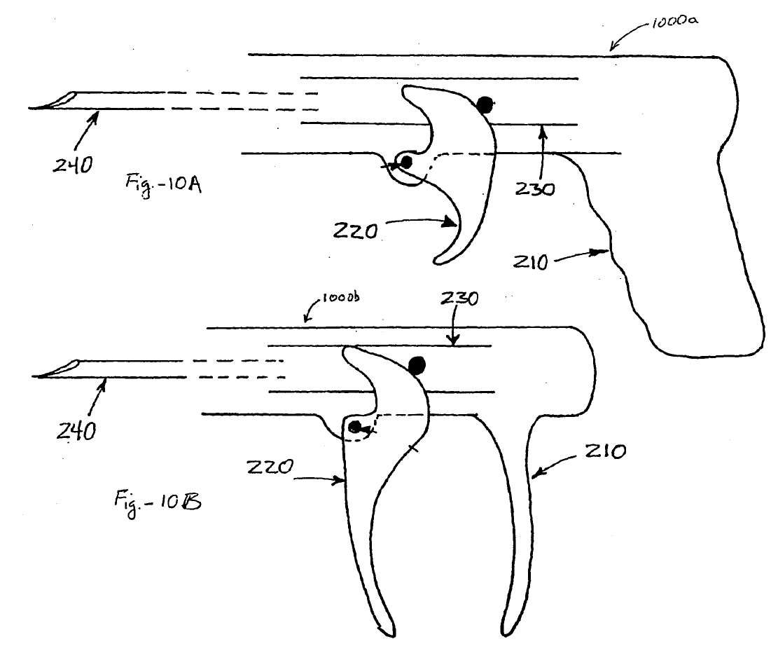

FIG. 11 is a cross-sectional view of an introducer 1100 in accordance with another embodiment of the invention. In the introducer 1100, the button 220 has a push member 1110 and the actuator 230 has a rack of teeth 1120. In operation, as the button 220 rotates downwardly about a pin 905, the push member 1110 engages one of the teeth 1120 and drives the actuator 230 backward (arrow B). The button 220 can be spring actuated such that it rotates upwardly under the influence of a spring (not shown). The actuator 230 can be driven incremenetally backwards with each subsequent depression of the button 220. The introducer 1100 further includes a cocking member 1130 in contact with the cannula 240 to restrict forward movement of the actuator 230. The cocking member 1130, more specifically, can be positioned in an opening 1140 of the handle and have a hole 1150 through which the cannula 240 passes.

One aspect of the introducer 1100 is that the teeth 1120 along the actuator 230 can be arranged such that a predetermined number of button depressions will drive the actuator 230 and cannula 240 backwards to release a marker from the cannula 240. As such, a plurality of markers can be loaded into the cannula 240 and each marker can be selectively ejected from the introducer by incrementally moving the actuator 230 backward by a set distance to controllably eject only a single marker at a time.

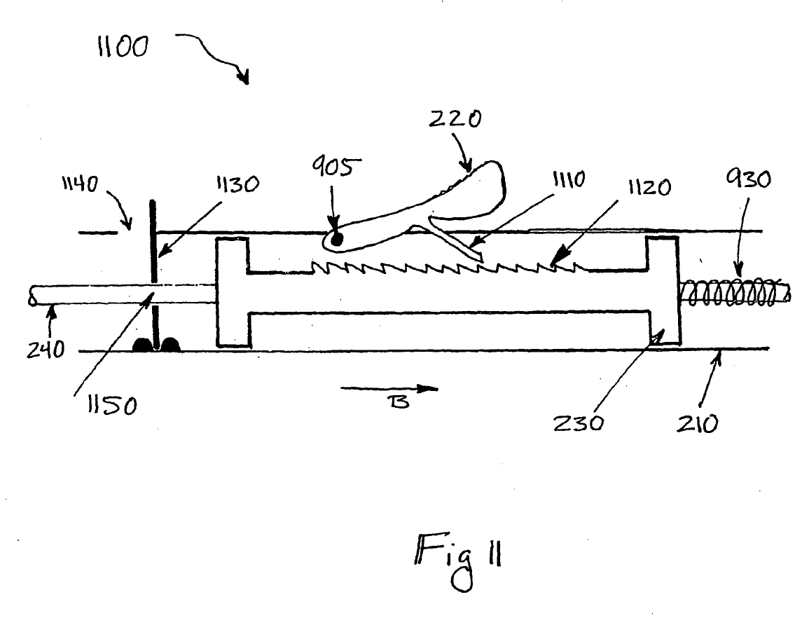

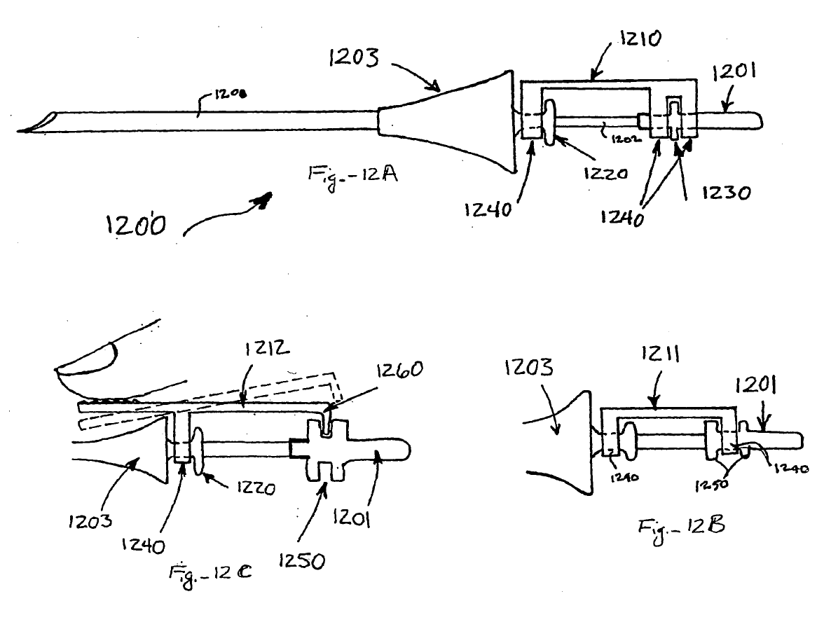

FIGS. 12A-C are cross-sectional views of introducers 1200 in accordance with additional embodiments of the invention. Like reference numbers refer to like components in FIGS. 1 and 12A-C. The introducer 1200 includes a first handle 1201, a stylet 1202 projecting from the first handle 1201, a second handle 1203 through which the stylet 1202 passes, and a cannula 1208 projecting from the second handle 1203 and receiving the stylet 1202. The introducer 1200 of FIG. 12A further includes a retainer 1210 that holds the first handle 1201 within a set distance from the second handle 1203. The retainer 1210 restricts the stylet 1202 from moving longitudinally with respect to cannula 1208. This in turn prevents inadvertently discharging a marker from the distal end of the cannula 1208 or from dislodging the stylet 1202 from the proximal end of the cannula 1208. The retainer 1210 can also be used to identify individual introducers and the markers contained within the introducers. For example, the retainer 1210 can have a specific color, shape, symbol, or indicia that indicates the frequency or other aspect of the marker contained in the introducer. In one embodiment, three markers having unique frequencies are loaded into three separate cannulas for implantation into the prostate gland. Each of the three retainers in this example can have suitable indicia to indicate that a first marker having a first resonant frequency is located in a first introducer, a second marker having a second resonant frequency is located in a second introducer, and a third maker having a third resonant frequency is loaded in a third introducer.

The embodiment of the retainer 1210 illustrated in FIG. 12A includes flexible C-clamps 1240 made from spring steel, plastic or other suitable elastic materials. The C-clamps 1240 are arranged so that one C-clamp engages the second handle 1203 next to a receiving collar 1220 and the other two C-clamps engage the first handle 1201 on both sides of a flange 1230. FIG. 12B illustrates an alternative embodiment having a retainer 1211 with a first end attached to the second handle 1203 and a second end having a single C-clamp 1240. In this embodiment, the first handle 1201 includes a collar 1250 that receives the C-clamp 1240. FIG. 12C illustrates an embodiment of a retainer 1212 having a first pivoting C-clamp 1240 coupled to the collar 1220 and a tab 1260 received by the collar 1250 on the first handle 1201. In operation, the retainer 1212 pivots from a lock position (shown in solid lines) to a release position (shown in broken lines) to allow for relative movement between the first handle 1201 and the second handle 1203.

FIG. 12D is an isometric view of an introducer 1200 in accordance with still another embodiment of the invention. In this embodiment, the introducer 1200 includes a retainer 1270 having a proximal section 1272 near the first handle 1201 and a distal portion 1274 near the second handle 1203. The retainer 1270 further includes a bulb 1276. The retainer 1270 is formed from a flexible material, such as silicone rubber, such that the bulb 1276 urges the first handle 1201 in a proximal direction away from the second handle 1203 until the bulb 1276 reaches an equilibrium position. In operation, as the second handle 1203 moves proximally towards the first handle 1201 to eject a marker (not shown) from the cannula 1208, the bulb 1276 folds over onto the proximal portion 1272 to allow relative movement between the second handle 1203 and the first handle 1201.

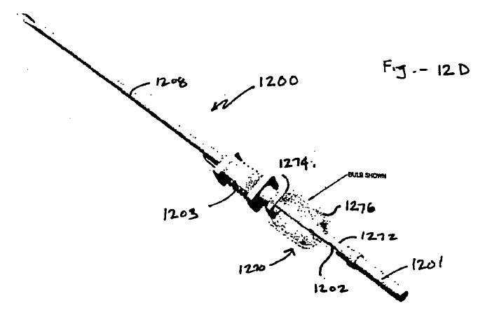

FIGS. 13-15 illustrate introducers in accordance with another aspect of the invention directed toward testing the markers while the markers are loaded in or otherwise assembled with the introducers. The markers typically have alternating magnetic transponders that wirelessly transmit a location signal in response to a wirelessly transmitted excitation energy. The markers, however, are not limited to alternating magnetic devices, but rather RF markers or other types of markers can also be tested using the introducers set forth in FIGS. 13-15.

FIG. 13 is a side view of an introducer 1300 in accordance with an embodiment of the invention directed to in-situ marker testing. In the embodiment shown in FIG. 13, the introducer 1300 includes a cannula 1302 having a marker holder 1303 configured to retain a marker 1304 so that a location signal 1305 from the marker 1304 propagates outside of the cannula 1302. The marker holder 1303, more specifically, can be a distal portion of the cannula 1302 having one or more openings 1310 (identified as a first opening 1310a and a second opening 1310b) in the cannula wall. The openings 1310 can be elongated holes or slots extending generally longitudinally along a portion of the length of the cannula 1302. In the embodiment illustrated in FIG. 13, the marker holder 1303 includes three openings 1310 spaced radially about the cannula 1302 at 120° increments.

The embodiment of the introducer 1300 operates by loading a marker 1304 into the cannula 1302 and holding the marker 1304 at the marker holder 1303. The marker 1304 can be held at the marker holder 1303 by a stylet (not shown) in the cannula 1302. The stylet can be held using a retainer as described above with reference to FIGS. 12A-D, or the stylet can have a large radius bend to create an interference fit between the stylet and the lumen of the cannula 1302. After loading the marker 1304, it can be tested by wirelessly transmitting an excitation energy at the resonant frequency of the marker 1304 and sensing the wirelessly transmitted location signal 1305 propagating from the marker 1304. The openings 1310 allow a sufficient amount of energy of the location signal 1305 to propagate outside of the cannula 1302 to be measured by a sensor. As such, the introducer 1300 enables in-situ testing of alternating magnetic markers or other types of active markers after they have been loaded into the introducer.

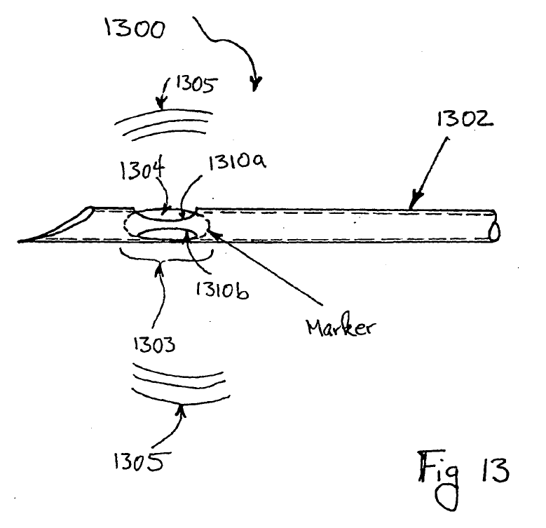

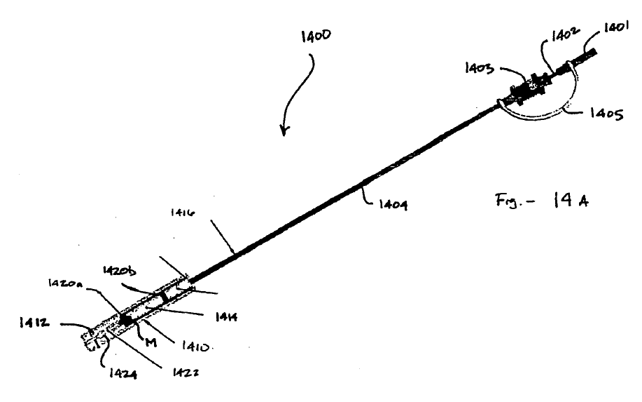

FIG. 14A is an isometric view of an introducer 1400 for in-situ testing of a marker M pre-loaded in the introducer 1400. In this embodiment, the introducer 1400 includes a first handle 1401, a stylet 1402 projecting from the first handle 1401, a second handle 1403 through which the stylet 1402 passes, and a cannula 1404 attached to the second handle 1403. The stylet 1402 passes through the second handle 1403 and is received in the lumen of the cannula 1404. The introducer 1400 can optionally include a tether attached to the first handle 1401 and the second handle 1403 to prevent the stylet 1402 from completely disengaging the cannula 1404.

The introducer 1400 further includes a marker holder 1410 at the distal portion of the cannula 1404. The marker holder 1410 includes a housing 1412 having a chamber 1414 and a sleeve 1416 projecting proximally from the housing 1412. The housing 1412 has a dielectric body composed of acrylics, polymers, or other suitable dielectric materials. The sleeve 1416 receives the cannula 1404 to guide the distal portion of the cannula 1404 into the chamber 1414. In this embodiment, the marker holder 1410 further includes a first retaining element 1420a in one portion of the chamber 1414, a second retaining element 1420b in a different portion of the chamber 1414, and a stop element 1422 projecting through an annulus 1424 at a distal portion of the marker holder 1410.

FIGS. 14B and 14C are cross-sectional views illustrating an embodiment of the marker holder 1410 in accordance with a specific embodiment of the invention. FIG. 14B, more specifically, illustrates the introducer 1400 in a testing configuration in which the marker M is loaded into the marker holder 1410 and the distal portion of the cannula 1404 is received in the cavity 1414. In this configuration, the first retaining element 1420a is a first O-ring or other resilient member that engages the exterior surface of the marker M and the second retaining element 1420b is a second O-ring or other type of resilient material that engages the outer surface of the cannula 1404. The marker M can be tested in this configuration by wirelessly transmitting an excitation energy through the housing 1412 and sensing a location signal S wirelessly transmitted from the marker M. Because the housing 1412 is composed of a dielectric material and the marker M is positioned just outside of the distal tip of the cannula 1404, the location signal S readily propagates through the marker holder 1410 to a sensor (not shown).

FIG. 14C illustrates the introducer 1400 in a loaded configuration after the marker M has been loaded into the cannula 1404. The marker M can be loaded into the cannula 1404 after it has been tested but before implanting the marker Into a patient. To load the marker M, the cannula 1404 is moved distally (arrow D) over the stylet 1402 and through the housing 1412. The distal portion of the cannula 1404 slides past the first retaining element 1420a until crimps at the distal tip of the cannula 1404 slide past the distal end of the marker M. Suitable crimps are described above with reference to crimps 444a and 444b in FIG. 4B. As the cannula 1404 progresses distally, the stop element 1422 holds the marker M in place and the annulus 1424 receives the distal portion of the cannula 1404. The marker holder 1410 can then be removed from the cannula 1404 without removing the marker M from the cannula 1404 because the crimps retain the marker M within the cannula 1404. After the marker holder 1410 is removed from the cannula 1404, the marker M is ready for implantation into a patient.

One advantage of the marker holder 1410 is that the dielectric housing does not significantly attenuate the strength of the marker signal S. Additionally, the marker holder 1410 protects the marker M and inhibits the marker M from inadvertently being ejected from the cannula 1404 until the patient is ready to have the marker M implanted. Moreover, the stylet 1402 can be pre-inserted into the cannula 1404 such that the entire introducer 1400 shown in FIG. 14A can be shipped as illustrated with a pre-tested and pre-loaded marker. This is expected to significantly reduce the likelihood that the marker will be ejected inadvertently and enhance the ease with which the marker M can be loaded into the cannula 1404.

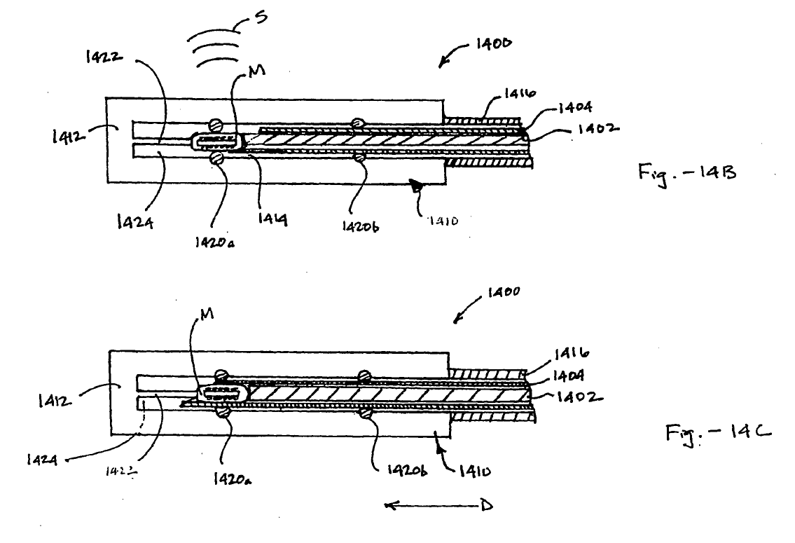

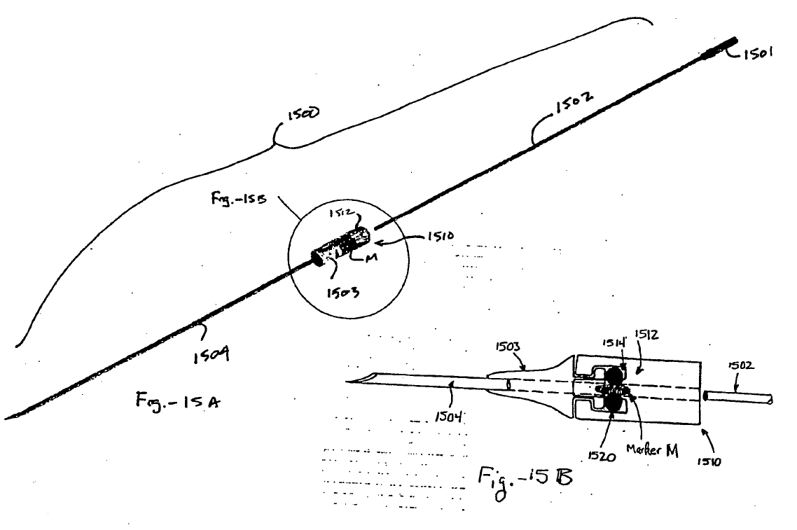

FIG. 15A is an exploded isometric view of an introducer 1500 for in-situ marker testing in accordance with yet another embodiment of the invention. In this embodiment, the introducer 1500 has a first handle 1501, a stylet 1502 projecting from the first handle 1501, a second handle 1503, and a cannula 1504 projecting from the second handle 1503. The introducer 1500 further includes a proximal marker holder 1510 having a dielectric housing 1512 that contains the marker M during a testing stage.

FIG. 15B is a cross-sectional view illustrating a specific embodiment of the marker holder 1510 in greater detail. In this embodiment, the dielectric housing 1512 includes a chamber 1514 in which the marker M is positioned for testing. The marker holder 1510 further includes a retaining element 1520 that engages the outer surface of the marker M to retain the marker M in the chamber 1514. The retaining element 1520 can be an O-ring or other type of resilient element, such as a polymeric material. In operation, the marker M is loaded into the marker holder 1510 and tested by exciting the marker with a wirelessly transmitted excitation energy. The location signal from the marker M propagates through the dielectric housing 1512 and is sensed by a sensor array. After the marker M has been tested, the stylet 1502 is passed through the marker holder 1510 and a portion of the cannula 1504 to drive the marker M through the cannula 1504 until it reaches a distal portion of the cannula. Unlike the introducer 1400 illustrated in FIG. 14A, the introducer 1500 inserts the stylet 1502 into the cannula 1504 after testing the marker M.

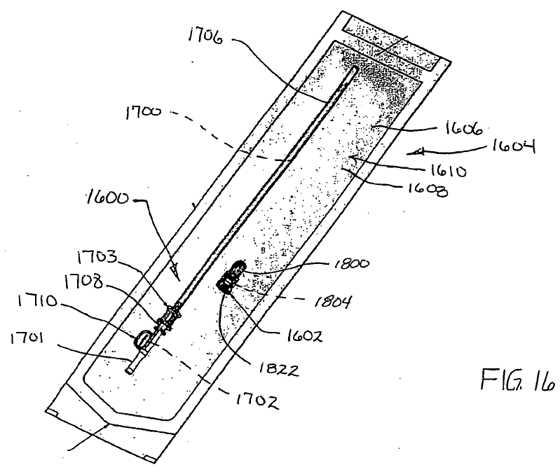

FIG. 16 illustrates an introducer 1600 and a marker container 1602 in a package assembly 1604 in accordance with another aspect of the invention. The package assembly 1604 provides a sterile, sealed environment that protects the introducer 1600 and the marker container 1602 from contamination. The package assembly 1604 and the marker container 1602 are configured to allow a marker 1804 to be tested in a manner (discussed above) after the marker 1804 and marker container 1602 have been packaged. The package assembly 1604 of the illustrated embodiment includes a backing sheet 1606 and a substantially transparent sheet 1604 hermitically sealed together generally around their perimeters to define an interior area 1610 that contains the introducer 1600 and the marker container 1602. In one embodiment, backing sheet 1606 is a substantially opaque sheet material, and the transparent sheet 1604 is Mylar®, although other materials could be used. The transparent sheet allows a user to visually inspect the introducer 1600 and marker container 1602 in the interior area 1610 without opening the package.

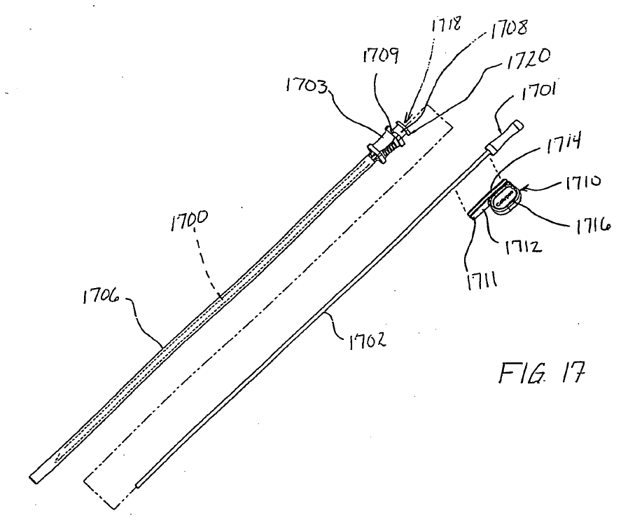

FIG. 17 is an enlarged, partially exploded isometric view of the introducer 1600 shown removed from the package assembly 1604 of FIG. 16. The introducer 1600 includes a first handle 1701, a stylet 1702 projecting from the first handle, a second handle 1703 through which the stylet passes, and a cannula 1700 projecting from the second handle and receiving the stylet. The introducer 1600 can also include a protective sheath 1706 that removably covers and protects the cannula 1700 until the introducer is ready for use.

The second handle 1703 of the illustrated embodiment includes a hub 1708 at a proximal end 1709. The hub 1708 has an opening 1718 through which the stylet 1702 passes as the stylet Is moved axially into the cannula 1700. The introducer 1600 of the illustrated embodiment also includes a stylet spacer 1710 removably connectable to the stylet 1702 adjacent to the first handle 1701. The stylet spacer 1710 restricts the stylet 1702 from moving too far into the cannula 1700 to prevent a marker (not shown) from being inadvertently discharged from the distal end of the cannula 1700.

The stylet spacer 1710 of the illustrated embodiment includes a generally cylindrical body 1712 with an elongated channel 1714 that removably receives a portion of the stylet 1702 adjacent to the first handle 1701. The body 1712 of one embodiment is made of a partially compressible material, such as a rubber, so the stylet spacer 1710 can allow the stylet 1702 to axially move a small amount relative to the cannula 1700 without damaging the components or discharging the marker. The channel 1714 is sized such that the body 1712 clips onto and frictionally engages the stylet 1701 to hold the stylet spacer 1710 in place on the stylet.

The stylet spacer 1710 is also configured so that a distal end 1711 of the body 1712 extends partially into the hub 1708 through the opening 1718 when the stylet 1702 is positioned in the cannula, as shown in FIG. 16. The distal end 1711 of the body 1712 is configured to frictionally engage the hub 1708 so that the stylet spacer 1710 also acts to prevent the stylet 1702 from being axially dislodged or inadvertently pulled out of the cannula 1700.

In the illustrated embodiment, the stylet spacer 1710 also includes a grip portion 1716 attached to the body 1712. The grip portion 1716 provides an area that a user can grip when removing the stylet spacer 1710 from the stylet 1702. In one embodiment, the grip portion 1716 is configured with an area that could include indicia, for example, to identify individual introducers and the markers contained within the introducers. In another embodiment, the stylet spacer 1710 can have a specific color, shape, symbol, or other indicia that indicates the frequency or other aspects of a marker contained in or associated with the introducer 1600, as discussed above.

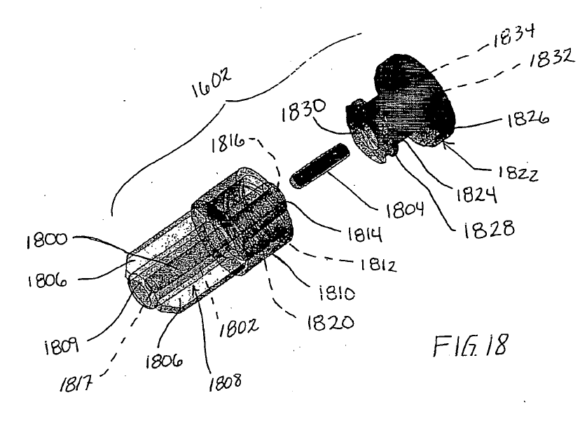

FIG. 18 is a partially exploded isometric view of a marker container 1602 in accordance with one embodiment of the invention. The marker container 1602 includes a container body 1800 with an interior chamber 1802 shaped and sized to contain a marker 1804. The container body 1800 also has an enlarged distal portion 1810 with an annulus 1812 that extends around a locking element 1814 projecting through the center of the annulus. In one embodiment, the locking element 1814 is a locking luer, although other releasable locking mechanisms could be used in other embodiments. The interior chamber 1802 of the illustrated embodiment is a blind hole that extends between an opening 1816 in the locking element 1814 and a closed end 1817 formed within the proximal portion 1809 of the container body 1800.

The interior chamber 1802 of the container body 1800 is sized to receive the marker 1804 through the opening 1816. The interior chamber 1802 of the illustrated embodiment has an inner diameter slightly greater than the exterior diameter of the marker 1804. Accordingly, the marker 1804 can slide axially within the interior chamber 1802, but is substantially restricted by the chamber walls from moving laterally within interior chamber. The container body 1800 in one embodiment is made of a dielectric material, such as parylene or another suitable dielectric material, that will allow the marker 1804 to be tested, as discussed above, when it is in the marker container 1650. The container body 1800 is also configured so it will not scratch the glass casing of the marker 1804.

The enlarged distal portion 1810 of the container body 1800 has internal threads 1820 within the annulus 1812. The annulus 1812 and internal threads 1820 removably receive a cap 1822 that screws into the annulus. In one embodiment, the cap 1822 is made of a dielectric material, such as parylene, that will not interfere with testing of the marker 1804 while it is in the marker container 1602.

The cap 1822 of the illustrated embodiment has a shaft 1824 with an enlarged knob 1826 on one end and external threads 1828 on the other end. The external threads 1828 are adapted to threadably mate with the internal threads 1820 in the annulus 1812. The shaft 1824 includes an aperture 1830 that receives the locking element 1814 when the cap is screwed onto the body portion. Accordingly, the shaft 1824 of the cap 1822 extends over the locking element 1814, covers the interior chamber 1802, and retains the marker 1804 in the container body 1800 until the cap 1822 is unscrewed and removed from the container body. In the illustrated embodiment, the internal and external threads 1820 and 1828 are conventional 6° locking luer threads that allow the cap to be quickly and easily removed from the chamber body 1800 to provide access to the locking element 1814.

In the illustrated embodiment, the container body 1800 is configured to allow a user to grasp the container body as the cap 1822 is unscrewed from the container body. In one embodiment, the container body 1800 has a gripping region 1808 formed by a pair of flanges 1806 project radially from a proximal portion 1809 of the container body 1800. Other embodiments can have container bodies 1800 with alternate configurations that allow a user to comfortably remove the cap 1822 from the container body.

In one embodiment, the knob 1826 of the cap 1822 has indicia 1832 on an exterior surface 1834 that can be used to identify the marker contained within the marker container 1800. In one embodiment, the indicia may be letters, numbers, symbols, colors or other indicia. In other embodiments, the cap 1822 can have different shapes or colors that provide an indication to a user which marker 1804 is contained in the marker container 1602.

The marker container assembly 1602 protects and isolates the marker 1804 until the marker 1804 is to be loaded into the introducer 1600 (FIG. 17). When the marker 1804 is to be loaded into the introducer 1600 (FIG. 17), a user grasps the gripping region 1808 and the knob 1826 and twists the cap 1822 to remove it from the chamber body 1800. After the cap 1822 is removed, the locking element 1814 and internal threads 1820 of the chamber body 1800 are exposed so that the chamber body can be connected directly to the hub 1708 on the second handle 1703 of the introducer 1600 (FIG. 17).

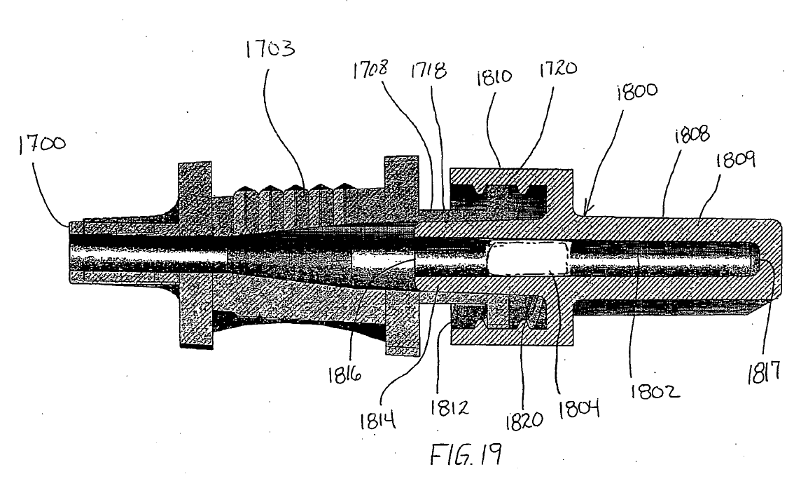

FIG. 19 is an enlarged cross-sectional view of the container body 1800 connected to the hub 1708 on the second handle 1703 of the introducer 1600. When the container body 1800 of the illustrated embodiment is attached to the hub 1708, the locking element 1814 extends into the opening 1718 in the hub 1708, and the hub fits the annulus 1812 in the container body. The flanges 1720 on the hub 1708 are sized to threadably mate with the interior threads 1820 of the container body 1800. Accordingly, the chamber body 1800 can be quickly and easily screwed onto the hub 1708 so that the interior chamber 1802 is axially aligned with the cannula 1700. The marker 1804 can then be transferred from the chamber body 1800 directly into the introducer 1600.

After the container body 1800 is attached to the hub 1708, the introducer 1600 and the container body 1800 can be oriented as a unit so that gravity drops the marker 1804 into the cannula 1700. The container body 1800 can then be removed from the hub 1708, and the stylet 1702 (FIG. 17) can then be moved through the hub 1708 and the second handle 1703. The stylet 1702 (FIG. 17) can then be inserted into the cannula 1700 to axially position the marker 1804 in a desired location within the cannula 1700 (FIG. 17). Although the illustrated embodiment shows a threaded engagement between the container body 1800 and the hub 1708 on the second handle 1703, other positive engagement mechanisms can be used to align the interior chamber 1802 of the container body 1800 with the cannula 1700 for delivery of the marker 1804 into the cannula 1700.

Although specific embodiments of, and examples for, the present invention are described herein for illustrative purposes, various modifications can be made without departing from the spirit and scope of the invention as will be readily apparent to those of ordinary skill in the relevant art. For example, although introducers are described above for implanting wireless active markers, the teachings of the present invention can also be applied to introducers for implanting markers that are hard-wired to a power source external to the patient. In these embodiments, for example, a suitable hole or other outlet can be provided in the introducer handle as required to accommodate passage of the wire. In addition, although the present disclosure describes manual introducers, in other embodiments, powered introducers that are at least partially automated can also be configured in accordance with embodiments of the present invention.

From the foregoing, it will be appreciated that specific embodiments of the invention have been described herein for purposes of illustration, but that various modifications may be made without deviating from the spirit and scope of the invention. Accordingly, the invention is not limited except as by the appended claims.

We claim:

1. A device for percutaneously implanting marker in a patient, the device comprising:
an elongated stylet having a proximal section and a distal section;
a first handle attached to the proximal section of the stylet;
a cannula having a proximal portion configured to receive the stylet and a distal portion configured to retain the marker;
a second handle attached to the proximal portion of the cannula; and
a retainer engaged with the first and the second handles, the retainer being configured to restrict relative movement between the first and second handles in a longitudinal direction.

2. The device of claim 1 wherein the retainer is configured to move between a first position in which the first handle is prevented from moving longitudinally relative to the second handle, and a second position in which the first and second handles can move longitudinally relative to each other.

3. The device of claim 2 wherein the retainer comprises a first C-clamp engaged with the first handle and second C-clamp engaged with the second handle, the first and second C-clamps being detachable from the first and second handles.

4. The device of claim 2 wherein:
the first handle has a first collar;
the second handle has a second collar; and
the retainer further comprises a first C-clamp engaged with the first handle next to the first collar, and a second C-clamp engaged with the second handle next to the second collar.

5. The device of claim 2 wherein the retainer comprises a tether having a first section attached to the first handle and a second section attached to the second handle.

6. The device of claim 2 wherein the retainer is pivotally attached to one of the first and second handles and the retainer contacts the other of the first and second handles in the first position.

7. The device of claim 2 where the retainer has a channel that removably receives the proximal section of stylet adjacent to the first handle.

8. The device of claim 2 wherein the retainer has a body portion with a channel that removably receives the stylet and a grip portion coupled to the body portion.

9. The device of claim 1 wherein the retainer comprises a flexible sleeve having a bulbous portion.

10. The device of claim 1, further comprising a marker having a unique characteristic loaded in the device, and wherein the retainer further comprises an indicator identifying the unique characteristic of the marker.

11. A device for implanting a marker into a patient, the marker having an alternating magnetic transponder that wirelessly transmits a location signal in response to a wirelessly transmitted excitation energy, the device comprising:
a cannula having a wall, a proximal portion, and a distal portion;
a marker holder at the proximal portion and/or the distal, portion of the cannula, wherein the marker holder is configured so that the location signal can propagate through the marker holder to a sensor.

12. The device of claim 11 wherein the marker holder comprises a testing region of the distal portion of the cannula, and the testing region has holes through the wall of the cannula.

13. The device of claim 12 wherein the holes comprise elongated slots through the wall of the cannula.

14. The device of claim 12 wherein the marker holder has three holes in the testing region arranged at 120° increments around the cannula.

15. The device of claim 11 wherein the marker holder comprises:
a dielectric housing having a chamber configured to receive the marker; and
a retaining element in the cavity for releasably retaining the marker in the cavity.

16. The device of claim 11 wherein the marker holder is carried at the distal portion of the cannula and the marker comprises:
a dielectric housing having a cavity configured to contain a marker and an opening in which the distal portion of the cannula is positioned; and
a first retaining element in the cavity.

17. The device of claim 16 wherein a marker is loaded in the marker holder such that the first retaining element contacts the marker.

18. The device of claim 17 wherein the marker holder further comprises a second retaining element in the cavity contacting the distal portion of the cannula.

19. The device of claim 18 wherein the first and second retaining elements comprise elastic members.

20. The device of claim 16 wherein the marker holder further comprises a stop element distally of the first retaining element and an annulus around at least a proximal portion of the stop element.

21. The device of claim 16 wherein the marker holder further comprises a sleeve extending proximally of the dielectric housing and over the cannula.

22. The device of claim 16 wherein the marker holder further comprises a second retaining element in the cavity contacting the distal portion of the cannula, a stop element distally of the first retaining element, an annulus around at least a proximal portion of the stop element, and a sleeve extending proximally of the dielectric housing and over a section of the cannula.

23. The device of claim 11 wherein the marker. holder is carried at the proximal end of the cannula and the marker holder further comprises:
a dielectric housing having a cavity configured to contain a marker and an opening configured to receive a stylet; and
a retaining element in the cavity for holding the marker.

24. The device of claim 23 wherein the marker is loaded in the marker holder such that the retaining element contacts the marker.

25. The device of claim 23 wherein the retaining element comprises a resilient member.

26. A device for implanting marker in a patient, the device comprising:
a cannula having an interior channel, a proximal portion, and a distal portion configured to retain the marker,
a handle attached to the proximal portion of the cannula, the handle having a hub in communication with the interior channel; and
a marker container having a connection portion and a chamber coupled to the connection portion, the connection portion being releasably coupleable to the hub with the chamber in communication with the interior channel to deliver the marker from the chamber into the interior channel.

27. The device of claim 26 wherein the marker container includes a closure device releasably attachable to the connection portion to close the chamber.

28. The device of claim 26 wherein the marker container has a threaded receiving area and a projection extending into the receiving area, at least a portion of the chamber extends through the projection, and the hub is configured to extend into the receiving area and threadably engage the marker container.

29. The device of claim 26 wherein the marker container has a annular receiving area and a locking luer projecting into the receiving area, at least a portion of the chamber extends through the locking luer, and the hub is configured to extend into the annular with the chamber and engage the locking luer.

30. A device for percutaneously Implanting an object in a patient, the device comprising:
a handle having an opening;
a stylet having a proximal section fixed to the handle, a medial section through the opening in the handle, and a distal section outside of the handle;
a cannula over at least a portion of the stylet, the cannula having a proximal portion slidably received through the opening of the handle and a distal portion configured to releasably hold the object; and
an actuator connected to the proximal portion of the cannula and received in the handle.

31. The device of claim 30 wherein the actuator further comprises a button configured to be pressed into the handle and an extension projecting from the button to limit the distance that the button can be pressed into the handle.

32. The device of claim 30, further comprising a cam member attached to the handle, and wherein the actuator further comprises a follower.

33. The device of claim 31 wherein the cam member comprises a button pivotally attached to the handle and the follower comprises an inclined surface on the actuator.

34. The device of claim 30, further comprising a button moveably attached to the handle and a plurality of teeth on the actuator, wherein the button is configured to engage the teeth to incrementally move the actuator proximally within the handle.

35. The device of claim 30 wherein the handle is configured to be held in a single hand of an operator, and wherein the actuator is configured to be manipulated by a digit of the single hand of an operator to slide the cannula relative to the handle and release the object within the patient.

36. The device of claim 30, further comprising an interface element coupled to the actuator, the interface element comprising a position selector that is manually operable to move the actuator from a first position to a second position, wherein the cannula at least generally retains the object when the actuator is in the first position, and wherein the cannula releases the object when the actuator is moved to the second position.

37. The device of claim 30 wherein the distal portion of the cannula includes a tip portion configured to percutaneously penetrate the patient, and wherein the tip portion is configured to releasably hold the object for implantation in the patient.

38. The device of claim 37 wherein the tip portion includes a restriction configured to releasably hold the object for implantation in the patient.

39. A device for percutaneously implanting an object in a patient, the device comprising:
a handle; and
a cannula having a wall and distal portion configured to releasably hold the object, wherein the cannula has a hole through the wall through which an electromagnetic signal can propagate.

40. The device of claim 39 wherein the hole is an elongated slot.

41. The device of claim 39 further comprising a plurality of holes arranged radially around the wall.

42. A device for holding a marker and loading the marker into a cannula, the device comprising:
a dielectric housing having a cavity and an opening at one end of the cavity; and
a resilient retaining element in the cavity having an inner diameter less than a diameter of the marker.

43. The device of claim 42 wherein the marker is in the cavity and the retaining element contacts the marker.

44. The device of claim 42 wherein the resilient member comprises an elastic O-ring.

45. The device of claim 42 wherein the opening is at a proximal end of the housing and the retaining element comprises a first retaining element at a distal portion of the cavity, and wherein the device further comprises a second retaining element at a proximal portion of the cavity.

46. The device of claim 42 wherein the opening is at a proximal end of the housing and the retaining element comprises a first retaining element at a distal portion of the cavity, and wherein the device further comprises:
a second retaining element at a proximal portion of the cavity;
a stop element distally of the first retaining element; and
an annulus around the stop element.

47. An assembly, comprising:
a marker having a casing and an alternating magnetic transponder in the casing, the transponder having a ferrite core and a coil wrapped around the core, and wherein the transponder wirelessly transmits a resonating location signal in response to a wirelessly transmitted excitation energy; and
a marker holder having a dielectric housing, a cavity in the housing in which the marker is received, an opening at a proximal end of the housing, and a retaining element in the cavity having a resilient contact surface contacting the casing of the marker.

48. The assembly of claim 47 wherein the resilient member comprises an elastic O-ring.

49. The assembly of claim 47 wherein the retaining element comprises a first retaining element at a distal portion of the cavity and the device further comprises a second retaining element at a proximal portion of the cavity.

50. The assembly of claim 47 wherein the retaining element comprises a first retaining element at a distal portion of the cavity and the device further comprises:
a second retaining element at a proximal portion of the cavity;
a stop element distally of the first retaining element; and
an annulus around the stop element.

51. A cannula for use in percutaneously implanting an object in, a patient, the cannula comprising a tube having a wall, a proximal inlet configured to receive the object, an intermediate portion extending distally from the proximal inlet, a distal portion extending distally from the intermediate portion, a tip with a cutting edge configured to penetrate the skin of the patient at the distal portion, a restriction at the cutting edge configured to releasably hold the object within the cannula, and a hole through the wall proximate to the distal tip to allow an electromagnetic signal to propagate from the cannula.

52. A method for percutaneously implanting an object in a patient, the method comprising:
restricting relative movement between a cannula and a stylet in a longitudinal direction with respect to a longitudinal axis of the cannula by engaging a retainer with the cannula and the stylet;
driving the cannula and the stylet into the patient while restricting relative longitudinal movement therebetween;
releasing the retainer to allow relative movement between the cannula and the stylet; and
drawing the cannula proximally over the stylet.

53. A method for handling a marker configured to be implanted into a patient, the marking having a transponder configured to wirelessly transmit a location signal in response to a wirelessly transmitted excitation energy, the method comprising:
loading the marker into a marker holder through which the location signal can propagate;
energizing the marker by wirelessly transmitting the excitation energy to the marker; and
sensing the location signal wirelessly transmitted from the marker through the marker holder.

54. The method of claim 53 wherein the marker holder comprises a dielectric housing carried at a proximal end of a cannula and the method further comprises loading the marker into the housing before energizing the marker and sensing the location signal.

55. The method of claim 54, further comprising inserting a stylet through the housing and the cannula after sensing the location signal to drive the marker through the cannula.

56. The method of claim 53 wherein the marker holder comprises a dielectric housing having a cavity containing the marker and a distal portion of a cannula is received in the cavity, and wherein the method further comprises positioning the distal portion of the cannula proximally of the marker such that at least a portion of the marker is outside of the cannula.

57. The method of claim 56 wherein the marker is energized and the location signal is sensed while at least a portion of the marker is outside of the cannula.

58. The method of claim 56, further comprising inserting a stylet into the cannula before energizing the marker.

59. The method of claim 58, further comprising shipping the marker while the marker is loaded in the cavity, the distal portion of the cannula is in the cavity, and the stylet is in the cannula.

60. The method of claim 56, further comprising removing the marker from the housing by moving the cannula distally so that restrictions at the distal portion of the cannula are located distally of the marker and then moving the cannula proximally until the distal portion of the cannula is withdrawn from the housing.

61. The method of claim 53 further comprising loading the market into a cannula having a wall, positioning the marker adjacent to a hole through the wall of the cannula, and propagating the location signal through the hole in the wall of the cannula.

62. A method for testing a marker and percutaneously implanting the marker in a patient, the method comprising:
energizing the marker while in a non-conducting container coupled to a proximal end and/or a distal end of a cannula;
loading the marker into the cannula;
driving cannula and the marker into the patient; and
moving the cannula in a proximal direction relative to a stylet in the cannula to release the marker within the patient.
